(19) United States
(12) Patent Application Publication
(10) Pub. No.: US 2015/0150801 A1
(43) Pub. Date: Jun. 4, 2015

(54) DOUBLE-LAYERED LIPOSOME COMPRISING INNER LIPOSOME COMPRISING HYDROPHOBIC ACTIVE INGREDIENT AND USE OF THE DOUBLE-LAYERED LIPOSOME

(71) Applicant: Samsung Electronics Co., Ltd., Suwon-si (KR)

(72) Inventors: Kitae PARK, Yongin-si (KR); Sang-joon PARK, Yongin-si (KR); Hyun-ryoung KIM, Guri-si (KR); Eun-sung PARK, Seongnam-si (KR)

(21) Appl. No.: 14/335,695

(22) Filed: Jul. 18, 2014

(30) Foreign Application Priority Data
Nov. 29, 2013 (KR) 10-2013-0147815

(51) Int. Cl. A61K 9/127; A61K 31/44

(52) U.S. Cl. CPC A61K 9/127; A61K 31/44

(57) ABSTRACT

A double-layered liposome comprising a first liposome comprising a hydrophobic active ingredient and a first lipid bilayer containing the hydrophobic active ingredient; and a second liposome comprising a second lipid bilayer having an inner space in which the first liposome is located; and a method of using the double-layered liposome.

### CROSS-REFERENCE TO RELATED APPLICATIONS

This application claims the benefit of Korean Patent Application No. 10-2013-0147815, filed on Nov. 29, 2013, in the Korean Intellectual Property Office, the entire disclosure of which is hereby incorporated by reference.

### BACKGROUND

1. Field

The present disclosure relates to double-layered liposomes including at least one inner liposome having a hydrophobic active ingredient, pharmaceutical compositions including the same, and methods of delivering the hydrophobic active ingredient to a target region in a body of a subject.

2. Description of the Related Art

A liposome includes at least one lipid bilayer surrounding an inner aqueous compartment. Liposomes are characterized by membrane types and sizes thereof. Small unilamellar vesicles (SUV) have a single layer and a diameter of about 20 nm to about 50 nm. Large unilamellar vesicles (LUV) have a diameter of 50 nm or greater. Oligolamellar vesicles and multilamellar vesicles have multiple, generally concentric, layers, and have a diameter of 100 nm or greater. A liposome having various non-concentric layers, i.e., a larger vesicle including smaller vesicles, refers to a multivesicular vesicle.

Liposomes may be formulated into therapeutic formulations, drugs, or other activating agents in which water-soluble active ingredients are distributed in an interior space and water-insoluble active ingredients are distributed in lipid bilayers.

The hydrophobic drugs may be delivered by using emulsions, co-solvents, micelles, and the like. Encapsulating a hydrophobic drug in the lipid bilayer of the liposome may affect the properties of the lipid bilayer, and thus, the stability or stimulus-sensitivity of the lipid bilayer may not be maintained. In addition, the hydrophobic drug encapsulated in the lipid bilayer may leak therefrom due to, for example, various blood proteins existing in blood.

Thus, there is still a need to develop liposomes capable of reducing the non-specific release of the hydrophobic drug in blood and controlling the release of the hydrophobic drug to be delivered to a target region in the body.

### SUMMARY

Provided is a double-layered liposome including a first liposome including a hydrophobic active ingredient and a first lipid bilayer containing the hydrophobic active ingredient, and a second liposome comprising a second lipid bilayer having an inner space in which the first liposome is contained.

Also provided is a pharmaceutical composition including the double-layered liposome, which is useful to deliver a hydrophobic active ingredient to a subject.

Further provided is a method for delivering a hydrophobic active ingredient to a target region of the body of a subject by administering a pharmaceutical composition including a double-layered liposome to the subject, and releasing the hydrophobic active ingredient by applying a stimulus to the target region in the body of the subject.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1A is a schematic diagram of a double-layered liposome;

FIG. 1B is a schematic diagram of drug release from a single-layered liposome or a double-layered liposome by blood protein;

FIG. 1C is a schematic diagram of drug release from a double-layered liposome by sonication;

FIG. 2A is a graph of sizes of inner liposomes;

FIGS. 2B and 2C are graphs of sizes of double-layered liposomes (FIG. 2B: before purification, FIG. 2C: after purification);

FIG. 3 is a graph of relative fluorescence intensities of single-layered liposomes and double-layered liposomes incubated in the presence of a BSA solution (open bars: single-layered liposome, hatched bars: double-layered liposome);

FIG. 4 is a graph of relative fluorescence intensities of double-layered liposomes plotted against sonication time;

FIG. 5 is a graph of sorafenib (%) released from double-layered liposomes plotted against sonication time; and

FIG. 6 is a graph of sorafenib (%) released from single-layered liposomes or double-layered liposomes incubated in the presence of a PBS buffer or a BSA solution (open bars: PBS buffer, hatched bars: BSA solution).

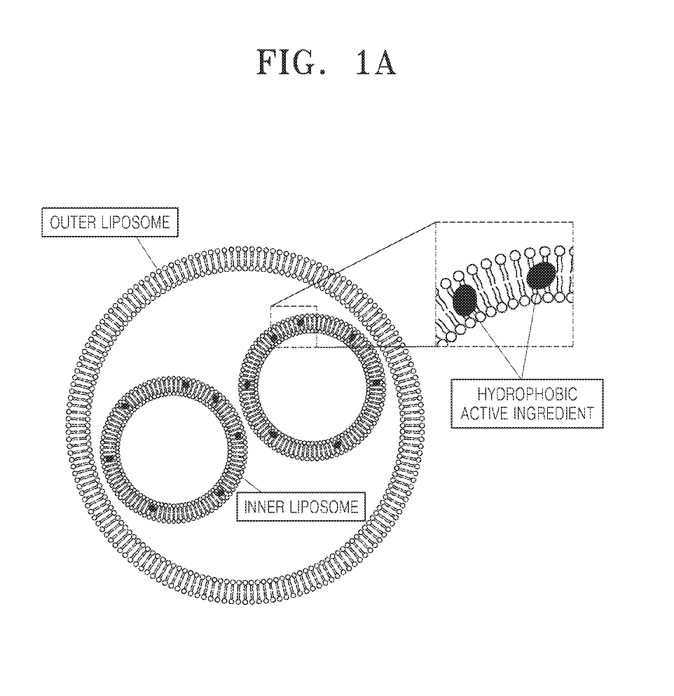

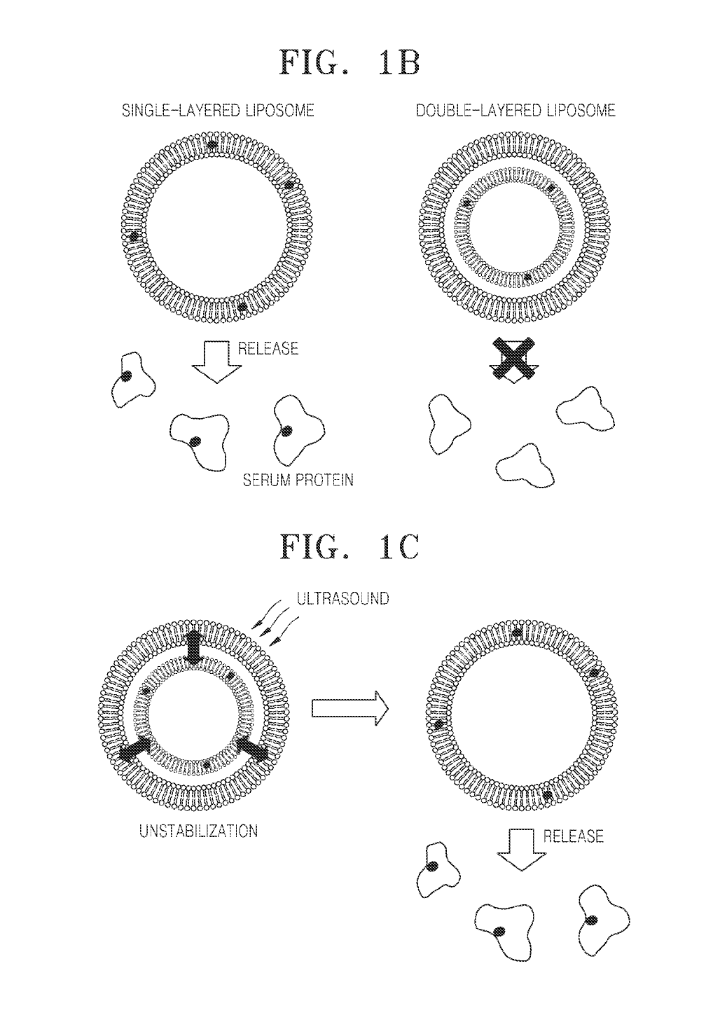

### DETAILED DESCRIPTION

Reference will now be made in detail to embodiments, examples of which are shown in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

A double-layered liposome according to an embodiment of the present invention includes: a first liposome including a hydrophobic active ingredient and a first lipid bilayer containing the hydrophobic active ingredient; and a second liposome including a second lipid bilayer having an inner space in which the first liposome is located.

The term “hydrophobic” means substantially unable to engage in attractive interactions with water molecules, and is associated with water-insoluble properties and non-polar properties. The term “hydrophobic” may be interchangeably used with a term “lipophilic”. Hydrophobic materials may be classified into the following groups according to water solubility: slightly soluble: 1 to 10 mg/ml; very slightly soluble: 0.1 to 1 mg/ml; substantially insoluble: <0.1 mg/ml.

The term “active ingredient” refers to a biologically active ingredient. The active ingredient may be a chemical compound, protein, peptide, nucleic acid, nano particle, or any combination thereof. The active ingredient may include anticancer drugs, angiogenesis inhibitors, anti-inflammatory agents, analgesics, anti-arthritic agents, sedatives, antidepressants, antipsychotic drugs, tranquilizers, anti-anxiety drugs, narcotic antagonists, anti-Parkinson drugs, cholinergic agonists, immunosuppressive drugs, antiviral agents, antibiotics, appetite suppressants, anticholinergic agonists, antihistamines drugs, anti-migraine drugs, hormone drugs, vasodiators, contraceptives, antithrombotic drugs, diuretics, antihypertensive agents, cardiovascular drugs, anti-wrinkle agents, anti-aging agents, skin whitening agents, or any combination thereof.

The hydrophobic active ingredient may be a hydrophobic drug, an imaging agent, or any combination thereof. The hydrophobic active ingredient may include a chemical substance or a biodrug having a water solubility of about 10 mg/ml or less. Examples of the hydrophobic active ingredient may include anthracycline-based materials, hydrophobic glucocorticoids, steroid-based substances, taxane-based drugs, cyclic peptide-based drugs, or any combination thereof. Examples of the anthracycline-based material may include doxorubicin, daunorubicin, epirubicin, idarubicin, valrubicin, mitoxantrone, or any combination thereof. Examples of the hydrophobic glucocorticoide may include Dexamethasone, Trimacinolone, Beclomethasone diproprionate, Triamcinolone acetonide, Triamcinolone diacetate, Bethamethasone diproprionate, Testosterone, Budesonide, 17α-ethinylestradiol, Levonorgestrel, Fluticasone proprionate, or any combination thereof. Examples of the hydrophobic active ingredient may include sorafenib, paclitaxel, docetaxel, doxorubicin, cyclosporine A, amphothericin B, indinavir, Rapamycin, Coenzyme Q10, Ursodeoxycholic acid, ilaprazole, Imatinib Mesilate, Tanespimycin, or any combination thereof. The imaging agent (or contrast media) refers to a material capable of improving contrast of an image by artificially increasing the difference of X-ray absorbance in each tissue during a medical test such as magnetic resonance imaging (MRI) or x-ray computed tomography (CT) scanning so as to efficiently recognize tissues or blood vessels. The imaging agent may be a transition element or a chelating complex of a transition element.

The term “lipid bilayer” refers to a membrane having two layers of lipid molecules. The lipid bilayer serves as a barrier that keeps ions, proteins, and other molecules where they are needed and prevent them from diffusing into areas where they should not be present. A lipid molecule forming the lipid bilayer is a molecule including a hydrophilic head and a hydrophobic tail. The lipid molecule may be a C₁₄-C₅₀ molecule.

The first lipid bilayer and the second lipid bilayer may have the same or different components. The first lipid bilayer and the second lipid bilayer may have the same or different composition ratios. The types of a stimulus for releasing the hydrophobic drug or a release rate of the hydrophobic drug may be controlled by changing components or composition ratio of the components of the lipid bilayer.

The first lipid bilayer and/or second lipid bilayer may include phospholipid, polyethylene glycol-conjugated lipid, cholesterol, or any combination thereof.

The phospholipid is a complex lipid having phosphoric acid ester in the molecules thereof. Phospholipid is a main component of all biomembranes such as cell membranes, endoplasmic reticulum, mitochondria, and myelin sheath surrounding nerve fibers. The phospholipid has a hydrophilic head and two hydrophobic tails. When phospholipid is exposed to water, the phospholipid is arranged into a double-layered sheet (a bilayer) with all of their tails pointing toward the center of the sheet. The center of this bilayer contains almost no water and excludes molecules like sugars or salts that dissolve in water but not in oil. The phospholipid may include phosphatidic acid, phosphatidylethanolamine, phosphatidylcholine, phosphatidylserine, phosphatidylinositol, phosphosphingolipid, or any combination thereof. The phosphoethanolamine (PE) may be 1,2-dioleoyl-sn-glycero-3-phosphoethanolamine (DOPE). The phosphatidylcholine (PC) may include a choline head group and glycerophosphoric acid tails. The glycerophosphoric acid may be a saturated or unsatrated fatty acid. The glycerophosphoric acid may be a C₁₄-C₅₀ molecule. The phosphatidylcholine may be 1,2-dierucoyl-sn-glycero-3-phosphocholine (DEPC), 1,2-distearoyl-sn-glycero-3-phosphocholine (DSPC), 1,2-dipalmitoyl-sn-glycero-3-phosphocholine (DPPC), egg phosphatidylcholine, soy phosphatidylcholine, or any combination thereof.

The polyethylene glycol (PEG)-conjugated lipid may be phosphatidylethanolamine (PE)-PEG. The PE may be a saturated fatty acid, an unsaturated fatty acid, a mixed acyl chain, lysophosphatidylethanolamine, or any combination thereof. The PEG-conjugated lipid may be 1,2-distearoylphosphatidylethanolamine-methyl-polyethylene glycol (DSPE-PEG).

The term “cholesterol” refers to a steroid compound. The term “cholesterol” includes cholesterol derivatives. Examples of the cholesterol derivatives may include sitosterol, ergosterol, stigmasterol, 4,22-stigmastadien-3-one, stigmasterol acetate, lanosterol, cycloartenol, or any combination thereof. The cholesterol may enhance the lipid bilayer and reduce permeability.

The “liposome” is an artificially prepared vesicle having a lipid bilayer. The liposome may be a unilamellar vesicle or a multivesicular vesicle. For example, the multivesicular vesicle may be a double-layered liposome including an inner liposome and an outer liposome. A multivesicular vesicle may contain an outer liposome and two or more inner liposomes.

The first liposome may be referred to as an inner liposome. The first liposome includes a hydrophobic active ingredient and a first lipid bilayer, and the hydrophobic active ingredient may be contained in the first lipid bilayer (e.g., within the lipid portion of the lipid bilayer, contained in an inner space defined by the lipid bilayer, or both).

The second liposome may be referred to as an outer liposome. The second liposome may include a second lipid bilayer, and the first liposome may be located in the inner space thereof (e.g., encapsulated by the second lipid bilayer). The inner space of the second liposome may constitute the inner area of the second lipid bilayer with respect to the center of the second lipid layer (e.g., the space encapsulated by the second lipid bilayer).

The first liposome and/or the second liposome may be a stimulus-sensitive liposome. The stimulus-sensitive liposome is a liposome that provides for controlled release of a material encapsulated in the liposome according to a stimulus applied thereto. The stimulus-sensitive liposome may include temperature-sensitive liposomes, pH-sensitive liposomes, chemosensitive liposomes, radiation-sensitive liposomes, ultrasound-sensitive liposomes, or any combination thereof. The temperature-sensitive liposomes, pH-sensitive liposomes, chemosensitive liposomes, radiation-sensitive liposomes, and ultrasound-sensitive liposomes may be liposomes respectively releasing a material encapsulated in each liposome under suitable environments or stimuli, for example, suitable temperature, suitable pH, existence of a chemical material, radiation, and ultrasound irradiation. The temperature at which encapsulated materials are released may be in the range of about 25° C. to about 70° C., about 30° C. to about 60° C., about 35° C. to about 50° C., or about 37° C. (body temperature) to about 50° C. The pH at which encapsulated materials are released may be, for instance, greater than about 7.5 or less than about 7.0, which is the pH of a saline solution. The chemical material includes materials that enable tumor cells to be more sensitive to chemotherapy. Example of the chemical material inducing release of an encapsulated material may include cyclosporine A, verapamil, biricodar, or any combination thereof. The radiation inducing release of an encapsulated material may be alpha (α)-rays, beta (β)-rays, gamma (γ)-rays, X-rays, or any combination thereof. The ultrasound inducing release of an encapsulated material may be sound waves having a frequency of about 16 Hz to about 20 kHz beyond the perceptive faculty of the human ear. The ultrasound may be high intensity focused ultrasound (HIFU). The HIFU refers to ultrasound having high intensity ultrasonic energy and focused on one area.

The first liposome may have a diameter of about 50 nm to about 300 nm, about 50 nm to about 250 nm, about 50 nm to about 200 nm, about 50 nm to about 150 nm, or about 100 nm to about 150 nm.

A liposome including the first and second liposomes may have a diameter of about 200 nm to about 1000 nm, about 250 nm to about 900 nm, about 300 nm to about 800 nm, about 350 nm to about 700 nm, about 350 nm to about 600 nm, or about 350 nm to about 500 nm.

A pharmaceutical composition to deliver a hydrophobic active ingredient according to an embodiment of the present disclosure includes a liposome. The liposome includes: at least one first liposome including a hydrophobic active ingredient and a first lipid bilayer containing the hydrophobic active ingredient; and a second liposome including a second lipid bilayer having an inner space in which the at least one first liposome is located. The liposome, hydrophobic, active ingredient, hydrophobic active ingredient, lipid bilayer, first lipid bilayer, first lipid bilayer, first liposome, second liposome, and interior space are as described above.

The pharmaceutical composition may further include a pharmaceutically acceptable carrier or diluent. Any known pharmaceutically acceptable carrier or diluent may be used. Examples of the carrier or diluent may include lactose, dextrose, sucrose, sorbitol, mannitol, starch, acacia rubber, calcium phosphate, alginate, gelatin, calcium silicate, micro-crystalline cellulose, polyvinyl pyrrolidone, cellulose, water (e.g., saline solution or sterile water), syrup, methylcellulose, methylhydroxy benzoate, propylhydroxy benzoate, talc, magnesium stearate, mineral oil, linger solution, buffer solution, maltodextrin solution, glycerol, ethanol, or any combination thereof. The pharmaceutical composition may further include a lubricant, a wetting agent, a sweetener, a flavoring agent, an emulsifying agent, a suspension agent, a preservative, or any combination thereof.

The pharmaceutical composition may be formulated with a pharmaceutically acceptable carrier and/or an excipient into a unit dosage form or a multiple dosage form by a well-known method in the art. In this regard, the formulation may be a solution in oil or an aqueous medium, a suspension, a syrup, an emulsifying solution, an extract, powder, granules, a tablet, or a capsule, and may further include a dispersing agent or a stabilizing agent. The aqueous medium may include a saline solution or a phosphate buffer solution (PBS).

A method for delivering a hydrophobic active ingredient to a target region in the body of a subject according to an embodiment of the present disclosure includes administering a pharmaceutical composition including a liposome to the subject, and releasing the hydrophobic active ingredient by applying a stimulus to the target region in the body of the subject. The liposome includes at least one first liposome including a hydrophobic active ingredient and a first lipid bilayer containing the hydrophobic active ingredient; and a second liposome including a second lipid bilayer having an inner space in which the at least one first liposome is located.

The method includes administering the pharmaceutical composition including the liposome to the subject. The liposome includes at least one first liposome including a hydrophobic active ingredient and a first lipid bilayer containing the hydrophobic active ingredient; and a second liposome including a second lipid bilayer having an inner space in which the at least one first liposome is located.

The liposome, hydrophobic, active ingredient, hydrophobic active ingredient, lipid bilayer, first lipid bilayer, first lipid bilayer, first liposome, second liposome, inner space, and pharmaceutical composition are as described above.

The subject may be a mammal such as a rat, mouse, dog, rabbit, horse, primate and human.

The administration may be an oral or parenteral administration. The parenteral administration may be performed via intravenous administration, subcutaneous administration, intramuscular administration, body cavity administration (intraperitoneal, intra-articular, or optical cavity administration), or direct injection. The direct injection may be an injection directly applied to an affected area, e.g., tumor area. The liposome may be administered into blood such as intravenously and may be delivered to a target region such as a tumor area via blood stream. The target region may be leaky such as an immature blood vessel in a tumor area. Dosage may variously be prescribed according to factors such as formulation method, administration method, age, weight, sex, and pathological condition of a patient, food, administration time, administration route, excretion rate, and reaction sensitivity. For example, the dosage may be in the range of about 0.001 mg/kg to about 100 mg/kg.

The method includes releasing the hydrophobic active ingredient by applying a stimulus to the target region in the body of the subject. The stimulus penetrates the target region of the body or otherwise contacts the liposome causing the liposome to release the encapsulated contents.

The subject is as described above.

The stimulus may be heating, changing pH, drug administration, radiation, ultrasound irradiation, or any combination thereof.

The heating may be performed by maintaining temperature in the range of about 25° C. to about 70° C., about 30° C. to about 60° C., about 35° C. to about 50° C., or about 37° C. (body temperature) to about 50° C. The heating may be performed by supplying heat for about 1 second to about 48 hours, about 1 minute to about 36 hours, about 5 minutes to about 24 hours, about 10 minutes to about 24 hours, about 30 minutes to about 12 hours, or about 1 hour to about 6 hours. The pH may be changed from about 5.5; that is the pH of a saline solution. The radiation may be alpha (α)-rays, beta (β)-rays, gamma (γ)-rays, X-rays, or any combination thereof. The ultrasound refers to sound waves having a frequency of about 16 Hz to about 20 kHz beyond the perceptive faculty of the human ear. The frequency of ultrasound may be in the range of about 20 kHz to about 2.0 MHz, about 20 kHz to about 1.5 MHz, about 20 kHz to about 1.0 MHz, about 20 kHz to about 500 kHz, or about 20 kHz to about 250 MHz. The ultrasound may be high intensity focused ultrasound (HIFU). The HIFU refers to ultrasound having high intensity ultrasonic energy and focused on one spot.

Due to the stimulus, the first lipid bilayer of the at least one first liposome and the second lipid bilayer of the second liposome are fused together, so that the hydrophobic active ingredient contained in the first liposome may be moved to the second liposome. The hydrophobic active ingredient moved to the second liposome may be released into blood by blood proteins.

The method may further include preventing or treating a disease by releasing the hydrophobic active ingredient from the liposome. The term “preventing” refers to inhibiting occurrence of disease. The term “treating” refers to inhibiting the progress of disease, or alleviating or removing disease. The disease may be cerebrospinal fluid tumor, head and neck cancer, lung cancer, breast cancer, thymoma, mesothelioma, esophagus cancer, gastric cancer, colorectal cancer, liver cancer, pancreatic cancer, bile duct cancer, kidney cancer, bladder cancer, prostate cancer, testicular cancer, germ cell tumors, ovarian cancer, cervical cancer, endometrial cancer, lymphoma, acute leukemia, chronic leukemia, multiple myeloma, sarcoma, malignant melanoma, skin cancer, or any combination thereof. The released hydrophobic active ingredient is absorbed by, or is placed into contact with, cells of the target region to show activity of the hydrophobic active ingredient.

According to the double-layered liposome including an inner liposome containing the hydrophobic active ingredient, the pharmaceutical composition including the same, and the method of delivering the hydrophobic active ingredient to the target region in the body of the subject using the double-layered liposome, stability of the liposome in the body may be improved since the hydrophobic active ingredient is contained in the first liposome and encapsulated by the second liposome, bioavailability of the hydrophobic active ingredient may be improved, and side effects may be reduced since normal tissues are protected by controlling drug release behavior and the released amount of the hydrophobic active ingredient using stimulus.

Embodiments of the present disclosure will be described in further detail with reference to the following examples. These examples are for illustrative purposes only and are not intended to limit the scope of the disclosure.

### EXAMPLE 1

### Preparation of Double-Layered Liposome Including Inner Liposome Containing Sorafenib and Measurement of Size of Double-Layered Liposome

1-1. Preparation of Inner Liposome Containing Sorafenib and Measurement of Size Thereof

1,2-dipalmitoyl-sn-glycero-3-phosphocholine (DPPC), cholesterol, 1,2-distearoyl-sn-glycero-3-phosphoethanolamine-N-[methoxy(polyethylene glycol)-2000 (ammonium salt) (DSPE-PEG), and sorafenib were mixed at a ratio of 55:4:2:1.1 (the ratio of sorafenib is 3 wt % of the main lipid) to prepare unilamellar vesicle inner liposomes.

Particularly, SA-V3-NH₂ (Peptron, Inc.) was dissolved in ethanol, and DPPC (Avanti Polar lipids, Inc.), cholesterol (Avanti Polar lipids, Inc.), DSPE-PEG (Avanti Polar lipids, Inc.), and sorafenib (Bayer) were dissolved in chloroform. The ethanol solution and the chloroform solution were mixed in a round-bottom flask, and the solvent was evaporated therefrom at room temperature using a rotary evaporator to form a lipid thin layer on an inner wall of the flask.

A 150 mM ammonium sulfate solution was added thereto at room temperature to hydrate the lipid thin layer. The hydrated solution was extruded using AVANTI® Mini-Extruder (Avanti Polar Lipids, Inc.) having a polycarbonate film having a pore size of 100 nm (Waters Corp.) to prepare unilamellar vesicle liposomes. The solvent of the prepared liposome solution was flowed through a PD-10 desalting column (GE Healthcare) while flowing the PBS to prepare inner liposomes containing sorafenib in the lipid bilayer. The prepared inner liposomes have a concentration of 10 mg/ml (based on the total lipid).

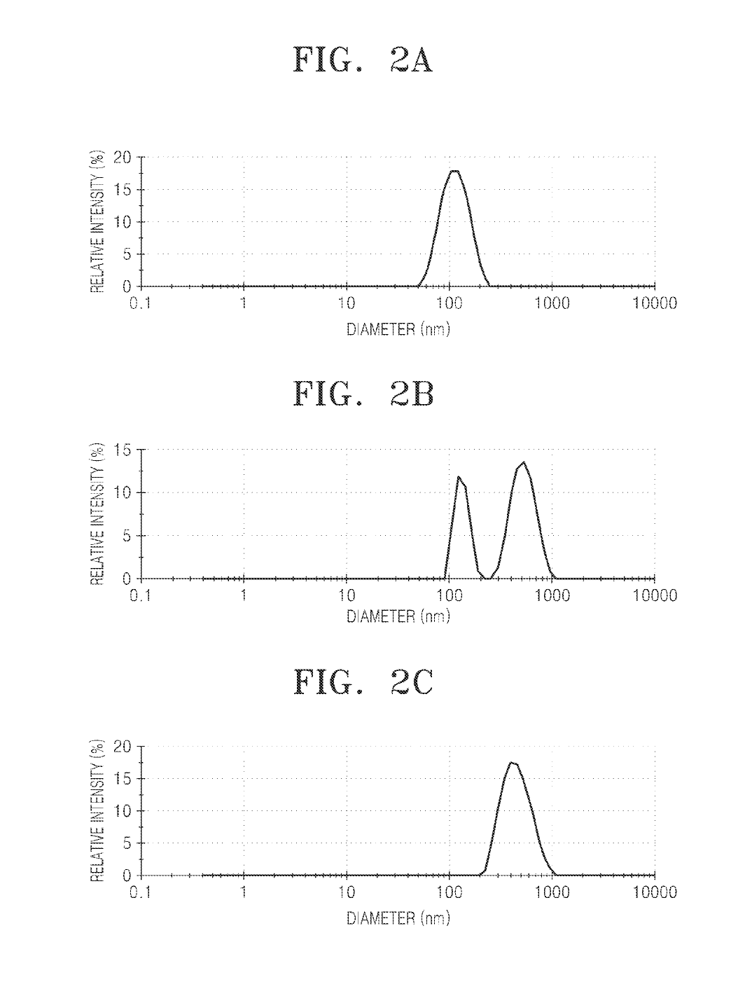

The prepared inner liposomes were analyzed using a dynamic light scattering (DLS) analyzer (Malvern Instruments Ltd.) to measure the size of the inner liposomes, and the results are shown in FIG. 2A. As shown in FIG. 2A, it was confirmed that the inner liposomes having a diameter of about 50 nm to 250 nm, and an average diameter of about 109.3 nm were prepared.

1-2. Preparation of Double-Layered Liposome Including Inner Liposome and Measurement of Size Thereof

SA-V3-NH₂ (Peptron, Inc.) was dissolved in ethanol. DPPC (Avanti Polar lipids, Inc.), cholesterol (Avanti Polar lipids, Inc.), and DSPE-PEG (Avanti Polar lipids, Inc.) were mixed in a ratio of 55:4:2, and the mixture was dissolved in chloroform. The ethanol solution and the chloroform solution were mixed in a round-bottom flask, and the solvent was evaporated therefrom at room temperature by using a rotary evaporator to form a lipid thin layer on an inner wall of the flask.

1 ml of the inner liposomes prepared as in Example 1-1 was added to the flask at room temperature to hydrate the lipid thin layer. The hydrated liposome solution was extruded using Avanti® Mini-Extruder (Avanti Polar Lipids, Inc.) having a polycarbonate film having a pore size of 500 nm (Waters Corp.) to prepare double-layered liposomes. The prepared double-layered liposomes were centrifuged at 4° C. for 20 minutes at a rate of 15,000×g, and pellets were re-suspended to purify the double-layered liposomes.

The double-layered liposomes were analyzed before and after purification using a dynamic light scattering (DLS) analyzer (Malvern Instruments, Ltd.) to measure the sizes of the liposomes, and the results are shown in FIGS. 2B and 2C. As shown in FIG. 2B, it was confirmed that liposomes before purification had a mixture of inner liposomes having an average diameter of about 136.2 nm and double-layered liposomes having an average diameter of about 469.9 nm. As shown in FIG. 2C, it was confirmed that the inner liposomes were removed after purification, and double-layered liposomes having a diameter of about 200 nm to 1000 nm and an average diameter of about 418.1 nm were prepared.

### EXAMPLE 2

### Identification of Stability of Double-Layered Liposome in Blood

In order to identify whether a hydrophobic material contained in double-layered liposomes is released in blood, a released amount of the hydrophobic material from the double-layered liposomes was measured in the presence of albumin that is a blood protein.

Single-layered liposomes were prepared in the same manner as in Example 1-1 except that 1,2-dioleoyl-sn-glycero-3-phosphoethanolamine (DOPE) was used instead of DPPC. Meanwhile, double-layered liposomes including a single-layered liposome were prepared in the same manner as Example 1-2, except that Nile Red (Aldrich), that is a hydrophobic dye, was used instead of sorafenib.

Bovine serum albumin (BSA) (Sigma Aldrich) was dissolved in distilled water to prepare a 100 mg/ml BSA solution.

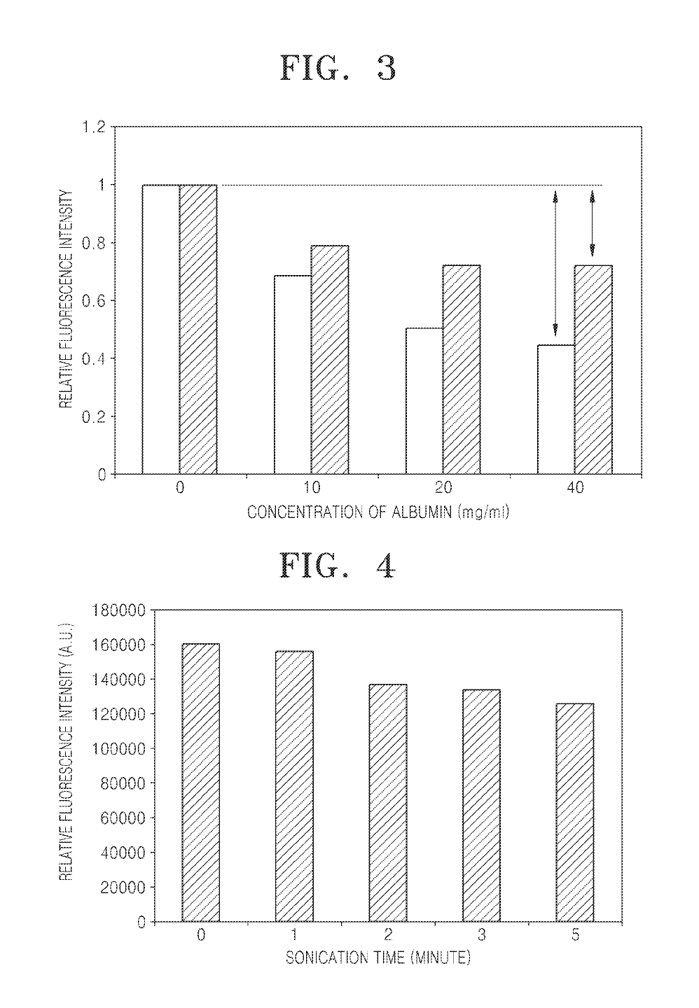

The single-layered liposomes or double-layered liposomes were mixed (or not mixed) mixed with a 10 mg/ml BSA solution, a 20 mg/ml BSA solution, or a 40 mg/ml BSA solution, and the mixtures were incubated at 37° C. for 10 minutes. Fluorescence intensities of the liposomes were measured at an excitation wavelength of 560 nm and an emission wavelength of 615 nm (PerkinElmer, Envision 2104-multilabel reader). When a fluorescence intensity of liposomes incubated in the absence of the BSA solution is considered to be 1, relative fluorescence intensities of liposomes are shown in FIG. 3 (open bars: single-layered liposome, hatched bars: double-layered liposome, ⇄: reduction in fluorescence intensity due to release of Nile Red).

As shown in FIG. 3, fluorescence intensity of liposomes was reduced since Nile Red is released from the liposomes by albumin. In addition, the fluorescence intensity of the double-layered liposomes was less reduced compared to the single-layered liposomes. Since the fluorescence intensity of the liposomes is reduced in accordance with the amount of Nile Red released from the liposomes, it was confirmed that release of the hydrophobic drug was suppressed in the double-layered liposomes when compared to the single-layered liposomes.

### EXAMPLE 3

### Release of Hydrophobic Material from Double-Layered Liposome by Ultrasound

It was identified whether the hydrophobic material contained in the inner liposome of the double-layered liposomes is released when the double-layered liposomes are exposed to ultrasound.

According to the method described above in Example 2, double-layered liposomes including inner liposomes in which Nile Red is contained were prepared.

The double-layered liposomes were mixed with a 40 mg/ml BSA solution, and the mixture was not sonicated or was sonicated at 4° C. for about 1 to about 5 minutes using a bath-type sonicator (Branson).

Fluorescence intensities of liposomes were measured at an excitation wavelength of 560 nm and an emission wavelength of 615 nm using Envision 2104-multilabel reader (PerkinElmer) and the measured fluorescence intensities thereof with respect to sonication time were shown in FIG. 4.

As shown in FIG. 4, fluorescence intensity of liposomes was reduced in proportion to the sonication time. Thus, it was confirmed that the amount of Nile Red released from the double-layered liposomes was increased in proportion to the sonication time.

### EXAMPLE 4

### Release of Sorafenib from Double-Layered Liposome by Ultrasound

It was identified whether sorafenib contained in the inner liposome of the double-layered liposomes is released when the double-layered liposomes are exposed to ultrasound.

Double-layered liposomes including an inner liposome in which sorafenib is contained were prepared according to the method as described above in Example 1.

500 μl of double-layered liposomes were mixed with a 40 mg/ml BSA solution, and the mixture was sonicated (or not sonicated) for 1, 3, or 5 minutes at 4° C. using a sonicator (Sonics and materials, Inc., VCX130) at a power of 130 watt, a wavelength of 20 kHz, and an amplitude of 50%.

The mixture exposed to ultrasound was incubated at 37° C. for 10 minutes, and then centrifuged at 4° C. for 10 minutes at a rate of 7,000×g to collect a supernatant, thereby obtaining albumin-bound sorafenib. The obtained albumin-bound sorafenib was quantified by high performance liquid chromatography (HPLC) to calculate a ratio (%) of sorafenib released from the double-layered liposome. The HPLC was performed using YMC-PACK C4 250×10 mm column (Waters Corp.) at a flow rate of 4 ml/min at 25° C. with a water/acetonitrile mobile phase at ratios of 100/0 (10 min), 30/70 (30 min), and 0/100 (11 min). Amounts of sorafenib released from the double-layered liposomes with respect to sonication time are shown in FIG. 5.

As shown in FIG. 5, the amount of sorafenib released from the double-layered liposomes increases in proportion to sonication time. About 97.5% of sorafenib was released from the double-layered liposomes during 5 minute sonication. Thus, it was confirmed that the amount of sorafenib released from the double-layered liposomes was increased in proportion to the sonication time.

### EXAMPLE 5

### Identification of Stability of Double-Layered Liposome

It was identified whether sorafenib is released from the inner liposome of the double-layered liposomes after incubating the double-layered liposomes in a PBS buffer or a BSA solution.

Single-layered liposomes containing sorafenib, and double-layered liposomes including an inner liposome having containing sorafenib were prepared according to the method shown in Example 1.

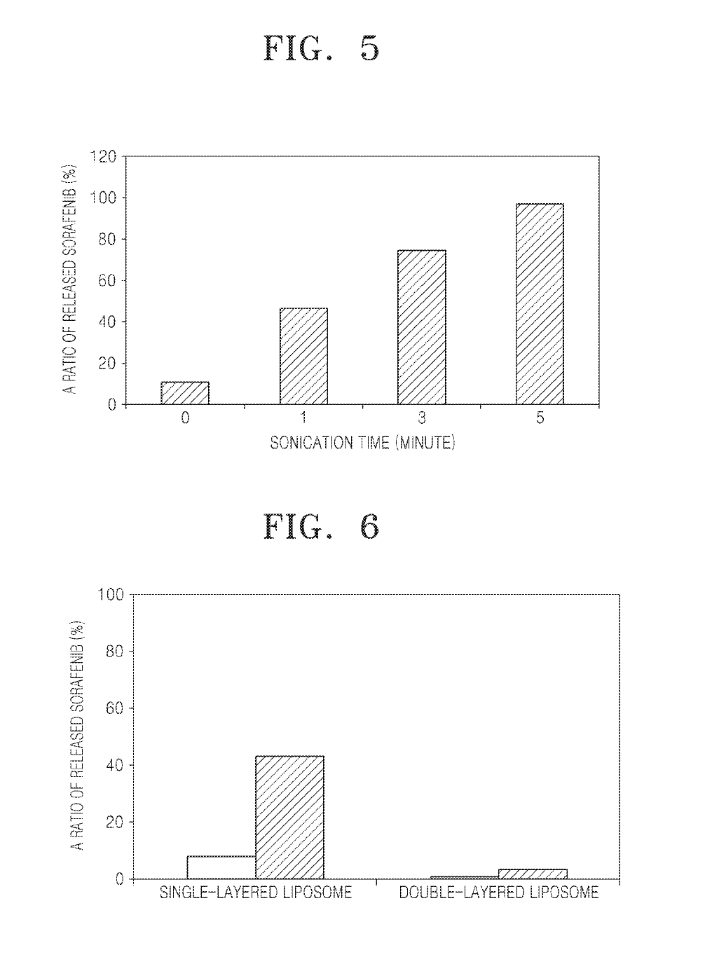

500 μl of the single-layered liposomes or 500 μl of the double-layered liposomes were mixed with a 1× PBS buffer or a 40 mg/ml BSA solution, and the mixture was incubated at room temperature for 72 hours. After the incubation, the mixture was centrifuged at 4° C. for 20 minutes at a rate of 200,000×g, and a supernatant was collected to obtain sorafenib released from the liposomes. The obtained sorafenib was quantified by HPLC. The HPLC was performed using YMC-PACK C4 250×10 mm column (Waters Corp.) at a flow rate of 4 ml/min at 25° C. with a water/acetonitrile mobile phase at ratios of 100/0 (10 min), 30/70 (30 min), and 0/100 (11 min). A ratio of the amounts of sorafenib (%) released from the single-layered liposomes or the double-layered liposomes were calculated in the presence of the 1× PBS buffer or the 40 mg/ml BSA solution, and the results are shown in FIG. 6 (open bars: PBS buffer, hatched bars: BSA solution).

As shown in FIG. 6, the amount of sorafenib released from the double-layered liposomes was less than that released from the single-layered liposomes. Thus, it was confirmed that the stability of the double-layered liposomes may be maintained for more than 24 hours in a living body, and a non-specific release of sorafenib that is a hydrophobic drug may be minimized.

It should be understood that the exemplary embodiments described therein should be considered in a descriptive sense only and not for purposes of limitation. Descriptions of features or aspects within each embodiment should typically be considered as available for other similar features or aspects in other embodiments. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A double-layered liposome comprising:
a first liposome comprising a hydrophobic active ingredient and a first lipid bilayer containing the hydrophobic active ingredient; and
a second liposome comprising a second lipid bilayer having an inner space in which the first liposome is located.

2. The double-layered liposome according to claim 1, wherein the hydrophobic active ingredient comprises a hydrophobic drug, an imaging agent, or a combination thereof.

3. The double-layered liposome according to claim 2, wherein the hydrophobic active ingredient comprises sorafenib, paclitaxel, docetaxel, doxorubicin, cyclosporine A, amphothericin B, indinavir, Rapamycin, Coenzyme Q10, Ursodeoxycholic acid, ilaprazole, Imatinib Mesilate, Tanespimycin, or any combination thereof.

4. The double-layered liposome according to claim 1, wherein the first lipid bilayer and the second lipid bilayer comprise the same components.

5. The double-layered liposome according to claim 1, wherein the first lipid bilayer and the second lipid bilayer comprise different components.

6. The double-layered liposome according to claim 1, wherein the first lipid bilayer or second lipid bilayer, or both, comprises phospholipid, polyethylene glycol-conjugated lipid, cholesterol, or any combination thereof.

7. The double-layered liposome according to claim 6, wherein the first lipid bilayer or second lipid bilayer, or both, comprises phosphatidic acid, phosphatidylethanolamine, phosphatidylcholine, phosphatidylserine, phosphatidylinositol, phosphosphingolipid, or any combination thereof.

8. The double-layered liposome according to claim 7, wherein the first lipid bilayer or second lipid bilayer, or both, comprises 1,2-dipalmitoyl-sn-glycero-3-phosphocholine (DPPC), 1,2-distearoyl-sn-glycero-3-phosphocholine (DSPC), egg phosphatidylcholine, soy phosphatidylcholine, or any combination thereof.

9. The double-layered liposome according to claim 1, wherein the first liposome or the second liposome, or both, is a stimulus-sensitive liposome.

10. The double-layered liposome according to claim 9, wherein the stimulus-sensitive liposome is a temperature-sensitive liposome, a pH-sensitive liposome, a chemosensitive liposome, a radiation-sensitive liposome, an ultrasound-sensitive liposome, or any combination thereof.

11. The double-layered liposome according to claim 1, wherein the first liposome has a diameter of about 50 nm to about 300 nm.

12. The double-layered liposome according to claim 1, wherein the double-layered liposome has a diameter of about 200 nm to about 1000 nm.

13. A pharmaceutical composition comprising a double-layered liposome to deliver a hydrophobic active ingredient to a subject,
wherein the double-layered liposome comprises:
a first liposome comprising a hydrophobic active ingredient and a first lipid bilayer containing the hydrophobic active ingredient; and
a second liposome comprising a second lipid bilayer having an inner space in which the first liposome is located.

14. A method for delivering a hydrophobic active ingredient to a target region in a subject, comprising:
administering a pharmaceutical composition comprising a double-layered liposome to the subject; and
releasing the hydrophobic active ingredient from the double-layered liposome by applying a stimulus to the target region in the body of the subject,
wherein the double-layered liposome comprises:
a first liposome comprising a hydrophobic active ingredient and a first lipid bilayer containing the hydrophobic active ingredient; and
a second liposome comprising a second lipid bilayer having an inner space in which the first liposome is located; and
wherein the first liposome, the second liposome, or both are stimulus-sensitive.

15. The method according to claim 14, wherein the subject is a mammal comprising a rat, mouse, dog, rabbit, horse, primate or human.

16. The method according to claim 14, wherein the stimulus comprises heat, pH change, drug administration, radiation, ultrasound irradiation, or any combination thereof.

17. The method according to claim 16, wherein the ultrasound has a frequency of about 20 kHz to about 2.0 MHz.
